(19) United States
(12) Patent Application Publication
(10) Pub. No.: US 2020/0222109 A1
(43) Pub. Date: Jul. 16, 2020

(54) ELECTROSURGICAL INSTRUMENTS

(71) Applicant: COVIDIEN LP, MANSFIELD, MA (US)

(72) Inventor: STEPHEN J. STAMM, WHEAT RIDGE, CO (US)

(21) Appl. No.: 16/244,245

(22) Filed: Jan. 10, 2019

(51) Int. Cl. A61B 18/14

(52) U.S. Cl. CPC A61B 18/1445; A61B 2018/00922

(57) ABSTRACT

A handle assembly includes a stationary handle having a first switch, a second switch, and a movable handle coupled thereto. The second switch is movable between a first position and a second position. The movable handle is movable relative to the stationary handle between a spaced position and an approximated position, in which the movable handle actuates the first switch. A portion of the movable handle is configured to contact the second switch when the second switch is in the first position to prevent the movable handle from actuating the first switch.

### FIELD

The present disclosure relates generally to the field of surgical instruments. In particular, the disclosure relates to endoscopic electrosurgical forceps.

### BACKGROUND

Instruments such as electrosurgical forceps are commonly used in open and endoscopic surgical procedures to coagulate, cauterize and seal tissue. Such forceps typically include a pair of jaw members that can be controlled by a surgeon to grasp targeted tissue, such as, e.g., a blood vessel. The jaw members may be approximated to apply a mechanical clamping force to the tissue, and are associated with at least one electrode to permit the delivery of electrosurgical energy to the tissue. The combination of the mechanical clamping force and the electrosurgical energy has been demonstrated to join adjacent layers of tissue captured between the jaw members. When the adjacent layers of tissue include the walls of a blood vessel, sealing the tissue may result in hemostasis, which may facilitate the transection of the sealed tissue.

A bipolar electrosurgical forceps typically includes opposed electrodes disposed on clamping faces of the jaw members. The electrodes are charged to opposite electrical potentials such that an electrosurgical current may be selectively transferred through tissue grasped between the electrodes. To affect a proper seal, particularly in relatively large vessels, the pressure applied to the vessel and the gap distance established between the electrodes are controlled.

### SUMMARY

The techniques of this disclosure generally relate to a mechanical block for preventing inadvertent manual actuation of an electrosurgical instrument.

In one aspect of the present disclosure, provided is a handle assembly including a stationary handle having a first switch, a second switch, and a movable handle coupled thereto. The second switch is movable between a first position and a second position. The movable handle is movable relative to the stationary handle between a spaced position and an approximated position. In the approximated position, the movable handle actuates the first switch. A portion of the movable handle is configured to contact the second switch when the second switch is in the first position to prevent the movable handle from actuating the first switch.

In aspects, the handle assembly may further include a protuberance extending proximally from the movable handle. The protuberance is the portion of the movable handle configured to contact the second switch when the second switch is in the first position to prevent the movable handle from actuating the first switch.

In aspects, the second switch may be configured to physically block the movable handle from moving to the approximated position when the second switch is in the first position.

In some aspects, the second switch may permit movement of the movable handle to the approximated position when the second switch is in the second position.

In further aspects, the second switch may be slidable relative to the stationary handle between the first and second positions.

In other aspects, the second switch may have a stop member facing the protuberance.

In aspects, the protuberance may move along a pathway during movement of the movable handle from the spaced position to the approximated position. The stop member may be aligned with the pathway of the protuberance when the second switch is in the first position, and out of alignment with the protuberance when the second switch is in the second position.

In some aspects, the first switch may include a depressible button attached to the stationary handle. The depressible button may be configured to be in electrical communication with an electrosurgical generator.

In further aspects, the movable handle may have a button activation post extending from a proximal side thereof, such that upon movement of the movable handle to the approximated position, the button activation post depresses the depressible button.

In other aspects, during movement of the movable handle toward the approximated position, the protuberance may be configured to contact the second switch prior to the button activation post contacting the depressible button.

In accordance with another aspect of the present disclosure, provided is an electrosurgical forceps. The electrosurgical forceps includes an end effector movable between an open configuration and a closed configuration, a shaft having a distal end coupled to the end effector, and a handle assembly coupled to a proximal end of the shaft. The handle assembly includes a stationary handle, first and second switches and a movable handle coupled to the stationary handle, and a protuberance extending proximally from the movable handle. The first switch is configured to transmit electrosurgical energy to the end effector, and the second switch is movable between a first position and a second position. The movable handle is movable relative to the stationary handle between a spaced position and an approximated position. In the approximated position, the movable handle actuates the first switch. The protuberance is configured to contact the second switch when the second switch is in the first position to prevent the movable handle from actuating the first switch.

In aspects, the movable handle may be operable to move the end effector between the open and closed configurations.

As is traditional, the term “distal” refers herein to an end of the electrosurgical forceps or component thereof that is farther from an operator, and the term “proximal” refers herein to the end of the electrosurgical forceps or component thereof that is closer to the operator.

### BRIEF DESCRIPTION OF THE DRAWINGS

Objects and features of the present disclosure will become apparent to those of ordinary skill in the art when descriptions of various embodiments thereof are read with reference to the accompanying drawings, of which:

FIG. 1 is a perspective view of an electrosurgical forceps according to an embodiment of the present disclosure including a handle assembly, an elongated shaft, and an end effector;

FIG. 2 is a side view illustrating a safety mechanism of the handle assembly of FIG. 1; and

FIG. 3 is an enlarged, perspective view illustrating details of the safety mechanism of FIG. 2.

### DETAILED DESCRIPTION

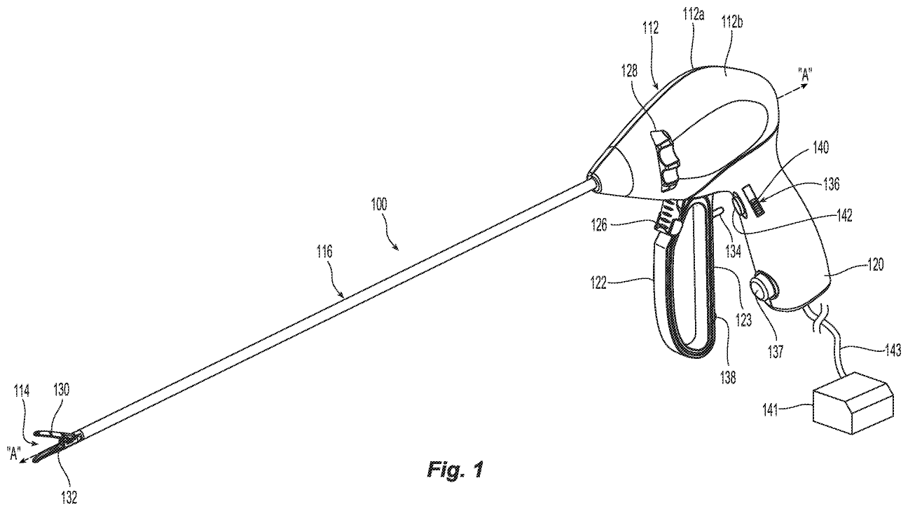

With reference to FIG. 1, an electrosurgical forceps 100 generally includes a handle assembly 112 that supports various actuators thereon for remotely controlling an end effector 114 through an elongated shaft 116. Although this configuration is typically associated with instruments for use in laparoscopic or endoscopic surgical procedures, various aspects of the present disclosure may be practiced with traditional open instruments and in connection with endoluminal procedures as well. The handle assembly 112 is constructed of a right housing half 112a and a left housing half 112b. The right and left designation of the housing halves 112a, 112b refer to the respective directions as perceived by an operator using the forceps 100. The housing halves 112a, 112b may be constructed of sturdy plastic, and may be joined to one another by adhesives, ultrasonic welding or other suitable assembly methods.

To mechanically control the end effector 114, the handle assembly 112 has a stationary handle 120, a movable handle 122, a trigger 126 and a rotation knob 128. The movable handle 122 is operable to move the end effector 114 between an open configuration wherein a pair of opposed upper and lower jaw members 130, 132 are disposed in spaced relation to one another, and a closed or clamping configuration wherein the jaw members 130, 132 are closer together. Approximation of the movable handle 122 with the stationary handle 120 moves the end effector 114 to the closed configuration and separation of the movable handle 122 from the stationary handle 120 moves the end effector 114 to the open configuration. The trigger 126 is operable to extend and retract a knife blade (not shown) through the end effector 114 when the end effector 114 is in the closed configuration. The rotation knob 128 rotates the elongated shaft 116 and the end effector 114 about a longitudinal axis A-A defined through the forceps 100.

To electrically control the end effector 114, the stationary handle 120 supports a first switch 137 thereon, such as, for example, a depressible button, which is operable by the user to initiate and terminate the delivery of electrosurgical energy to the end effector 114. The first switch 137 is engageable by a button activation post 138 extending from a proximal side 123 of the moveable handle 122 upon proximal movement of the moveable handle 122 to an actuated or approximated position. The first switch 137 is in electrical communication with an electrosurgical generator 141 via suitable electrical wiring (not explicitly referenced) extending from the housing 112 through a cable 143 extending between the housing 112 and the electrosurgical generator 141. The generator 141 may include devices such as the LigaSure® Vessel Sealing Generator and the ForceTriad® Generator sold by Covidien. The cable 143 may include a connector (not shown) thereon such that the forceps 100 may be selectively coupled electrically to the generator 141.

For a more detailed description of various features of the electrosurgical forceps 100, reference may be made to U.S. Pat. No. 9,655,673, the entire contents of which incorporated by reference herein.

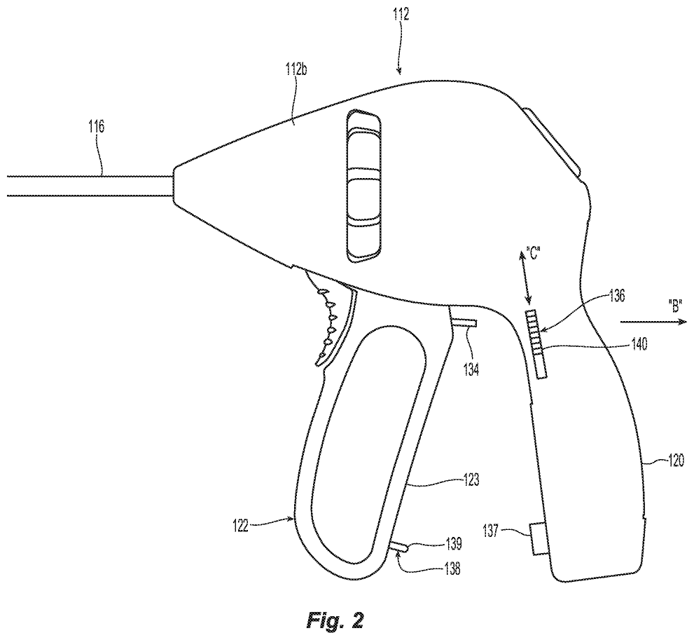

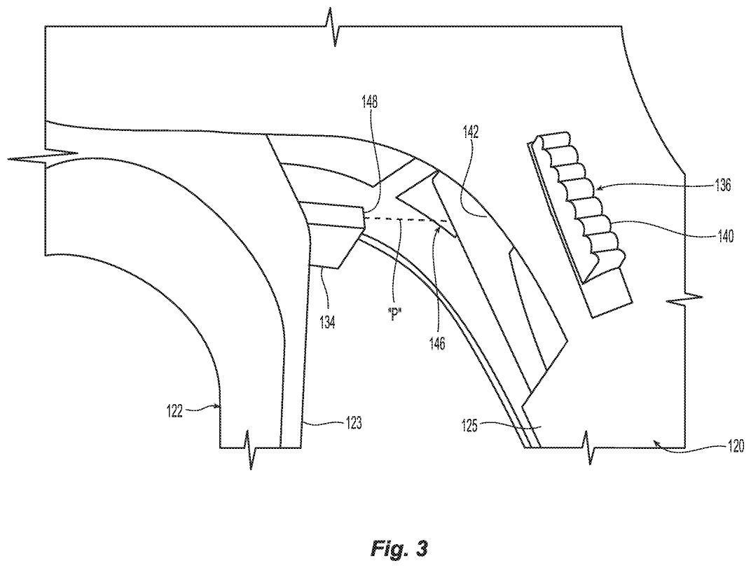

With reference to FIGS. 2 and 3, the handle assembly 112 has a safety mechanism that includes a second switch 136 and a protuberance 134 configured to interact with one another to selectively prevent actuation of the first switch 137. The second switch 136 is slidably coupled to the left housing half 112b or the right housing half 112a, and the protuberance 134, such as, for example, a post, is coupled to the movable handle 122. The second switch 136 has a slider 140 and a stop member 142 fixedly coupled to the slider 140. The slider 140 is disposed outside of the housing halves 112a, 112b, and therefore manually accessible by a user's hand. The second switch 136 is movable relative to the stationary handle 120 between a first or blocking position, and a second or unblocking position. In aspects, the slider 140 may be axially movable, rotatable, depressible, or movable relative to the stationary handle 120 in any suitable manner. In some aspects, the second switch 136 may be movable relative to the left housing half 112b or the right housing half 112a in any suitable manner, such as, for example, rotationally.

The stop member 142 of the second switch 136 faces the proximal side 123 of the movable handle 122 and is disposed within one or both of the housing halves 112a, 112b. The stop member 142 is a structural element (e.g., a flange) that resists being moved proximally (i.e., in the direction indicated by arrow “B” in FIG. 2). The stationary handle 120 has an access opening 146 defined in a distal side 125 thereof (FIG. 3). The stop member 142 of the second switch 136 is disposed behind or within the access opening 146 to provide the protuberance 134 of the movable handle 122 access to the stop member 142 of the second switch 136. The stop member 142 of the second switch 136 is aligned with a pathway “P” (FIG. 3) of the protuberance 134 when the second switch 136 is in the first position. Oppositely, when the second switch 136 is in the second position, the stop member 142 of the second switch 136 is out of alignment with the pathway “P” of the protuberance 134. The protuberance 134 may be a post. In other aspects, the proximal side 123 of the movable handle 122 may directly contact the second switch 136 rather than using the protuberance 134.

The protuberance 134 of the movable handle 122 extends proximally from the proximal side 123 of the movable handle 122 and is located at an upper portion of the movable handle 122 (e.g., above the activation post 138). The protuberance 134 is fabricated from a rigid material, such as a hard plastic, and may assume any suitable shape, such as, for example, a rectangle, a cone, a sheet, a cylinder, or the like. During approximation of the movable handle 122, the protuberance 134 is configured to contact the second switch 136 prior to the activation post 138 contacting the first switch 137 due to a proximal end 148 of the protuberance 134 being disposed proximally in relation to a proximal end 139 of the activation post 138. In some aspects, the spacing between the protuberance 134 and the second switch 136 may be less than the spacing between the activating post 138 and the first switch 137 due at least in part to the protuberance 134 having a greater length than the activation post 138. In other aspects, the spacing between the protuberance 134 and the second switch 136 may be less than the spacing between the activating post 138 and the first switch 137 due at least in part to the angled orientation of the movable handle 122 relative to the stationary handle 120. In other aspects, the proximal side 123 of the movable handle 122 may directly contact the first switch 137 rather than using the activation post 138.

In use, a clinician may wish to clamp and seal tissue between the jaw members 130, 132 of the end effector 114. To move the end effector 114 from the open configuration to the closed configuration, the movable handle 122 is moved generally proximally, in the direction indicated by arrow “B,” from a spaced position toward the approximated position. If the second switch 136 is in the first or blocking position, the protuberance 134 on the movable handle 122 contacts the stop member 142 of the second switch 136 prior to the movable handle 122 reaching the fully approximated position. Upon the protuberance 134 engaging the stop member 142 of the second switch 136, a further application of a clamping force on the movable handle 122 will fail to move the movable handle 122 closer to the approximated position. Since the engagement between the protuberance 134 and the stop member 142 prevents completion of the movement of the movable handle 122 to the approximated position, the activation post 138 of the movable handle 122 remains distally spaced from the first switch 137. Accordingly, so long as the second switch 136 remains in the first or blocking position, the activation post 138 fails to actuate the first switch 137 and electrosurgical energy is not transferred from the electrosurgical generator 141 to the end effector 114.

To actuate the first switch 137 and transmit electrosurgical energy to the end effector 114, the second switch 136 is moved from the first position to the second or unblocking position (e.g., via sliding the slider 140 along an axis indicated by arrow “C” in FIG. 2). As the second switch 136 is moved to the second position, the stop member 142 of the second switch 136 is cleared from the pathway “P” of the protuberance 134. With the pathway “P” cleared, the movable handle 122 is free to move to the fully approximated position, whereby the activation post 138 of the movable handle 122 simultaneously or substantially simultaneously contacts the first switch 137. Upon actuating the first switch 137 with the activation post 138, electrosurgical energy is transmitted from the electrosurgical generator 141 to the end effector 114 to seal the tissue disposed between the jaw members 130, 132.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A handle assembly, comprising:
a stationary handle;
first and second switches coupled to the stationary handle, the second switch movable between a first position and a second position; and
a movable handle coupled to the stationary handle and movable relative to the stationary handle between a spaced position and an approximated position, in which the movable handle actuates the first switch, wherein the movable handle has a portion configured to contact the second switch when the second switch is in the first position to prevent the movable handle from actuating the first switch.

2. The handle assembly according to claim 1, further comprising a protuberance extending proximally from the movable handle, wherein the protuberance is the portion of the movable handle configured to contact the second switch when the second switch is in the first position to prevent the movable handle from actuating the first switch.

3. The handle assembly according to claim 2, wherein the second switch has a stop member facing the protuberance.

4. The handle assembly according to claim 3, wherein the protuberance moves along a pathway during movement of the movable handle from the spaced position to the approximated position, the stop member aligned with the pathway of the protuberance when the second switch is in the first position, and out of alignment with the protuberance when the second switch is in the second position.

5. The handle assembly according to claim 1, wherein the second switch is configured to physically block the movable handle from moving to the approximated position when the second switch is in the first position.

6. The handle assembly according to claim 5, wherein the second switch permits movement of the movable handle to the approximated position when the second switch is in the second position.

7. The handle assembly according to claim 1, wherein the second switch is slidable relative to the stationary handle between the first and second positions.

8. The handle assembly according to claim 1, wherein the first switch includes a depressible button attached to the stationary handle and configured to be in electrical communication with an electrosurgical generator.

9. The handle assembly according to claim 8, wherein the movable handle has a button activation post extending from a proximal side thereof, such that upon movement of the movable handle to the approximated position, the button activation post depresses the depressible button.

10. The handle assembly according to claim 9, wherein during movement of the movable handle toward the approximated position, the portion of the movable handle is configured to contact the second switch prior to the button activation post contacting the depressible button.

11. An electrosurgical forceps, comprising:
an end effector movable between an open configuration and a closed configuration;
a shaft having a distal end coupled to the end effector; and
a handle assembly coupled to a proximal end of the shaft and including:
a stationary handle;
first and second switches coupled to the stationary handle, the first switch configured to transmit electrosurgical energy to the end effector, and the second switch movable between a first position and a second position;
a movable handle coupled to the stationary handle and movable relative to the stationary handle between a spaced position and an approximated position, in which the movable handle actuates the first switch; and
a protuberance extending proximally from the movable handle, wherein the protuberance is configured to contact the second switch when the second switch is in the first position to prevent the movable handle from actuating the first switch.

12. The electrosurgical forceps according to claim 11, wherein the second switch is configured to physically block the movable handle from moving to the approximated position when the second switch is in the first position.

13. The electrosurgical forceps according to claim 11, wherein the second switch permits movement of the movable handle to the approximated position when the second switch is in the second position.

14. The electrosurgical forceps according to claim 11, wherein the second switch is slidable relative to the stationary handle between the first and second positions.

15. The electrosurgical forceps according to claim 11, wherein the second switch has a stop member facing the protuberance.

16. The electrosurgical forceps according to claim 15, wherein the protuberance moves along a pathway during movement of the movable handle from the spaced position to the approximated position, the stop member aligned with the pathway of the protuberance when the second switch is in the first position, and out of alignment with the protuberance when the second switch is in the second position.

17. The electrosurgical forceps according to claim 11, wherein the first switch includes a depressible button attached to the stationary handle and configured to be in electrical communication with an electrosurgical generator.

18. The electrosurgical forceps according to claim 17, wherein the movable handle has a button activation post extending from a proximal side thereof, such that upon movement of the movable handle to the approximated position, the button activation post depresses the depressible button.

19. The handle assembly according to claim 18, wherein during movement of the movable handle toward the approximated position, the protuberance is configured to contact the second switch prior to the button activation post contacting the depressible button.

20. The electrosurgical forceps according to claim 11, wherein the movable handle is operable to move the end effector between the open and closed configurations.
